(19) United States
(12) Patent Application Publication
(10) Pub. No.: US 2010/0159573 A1
(43) Pub. Date: Jun. 24, 2010

(54) MICROFLUIDIC DILUTION DEVICE

(75) Inventors: Kwang Hyo CHUNG, Daejeon (KR); JuHyun Jeon, Daejeon (KR); Dae-Sik Lee, Daejeon (KR); Moon Youn Jung, Daejeon (KR); Seon Hee Park, Daejeon (KR)

Correspondence Address: AMPACC Law Group, 3500 188th Street S.W., Suite 103, Lynnwood, WA, 98037, US

(73) Assignee: Electronics and Telecommunications Research Institute, Daejeon (KR)

(21) Appl. No.: 12/534,012

(22) Filed: Jul. 31, 2009

(30) Foreign Application Priority Data
Dec. 22, 2008 (KR) 10-2008-0131295

(51) Int. Cl. C12M 1/00; B01L 3/00; G01N 33/48

(52) U.S. Cl. 435/287.1; 422/99; 422/68.1

(57) ABSTRACT

Provided is a microfluidic dilution device that uses capillary force to dilute first and second fluids in a predetermined ratio. The microfluidic dilution device includes a channel plate, a cover plate, fluid chambers, and a confluence chamber. The fluid chambers are filled with first and second fluids in a predetermined ratio. First and second fluids flowing to the confluence chamber are diluted in a predetermined ratio.

### CROSS-REFERENCE TO RELATED APPLICATIONS

This U.S. non-provisional patent application claims priority under 35 U.S.C. §119 of Korean Patent Application No. 10-2008-0131295, filed on Dec. 22, 2008, the entire contents of which are hereby incorporated by reference.

### BACKGROUND OF THE INVENTION

The present invention disclosed herein relates to a microfluidic dilution device configured to mix microfluids in a predetermined ratio, and more particularly, to a microfluidic dilution device using capillary force to dilute first and second fluids in a predetermined dilution ratio.

Microfluidic control chips are widely used to perform biochemical reactions on chips. Various microfluid-controlling operations, including mixing, diluting, transferring, branching, separating, and washing, are performed on a microfluidic control chip. Particularly, fluids such as reaction solutions, samples, and buffers may be mixed on a chip, or samples may be mixed or diluted in a predetermined ratio.

Typically, such mixing and diluting operations are performed outside a microfluidic control chip before samples are supplied into the chip, or performed by a mixer and a dilutor that are disposed on the microfluidic control chip, but controlled by an external controller. Devices controlled by an external controller are categorized as active devices, and devices without the external controller are categorized as passive devices. Particularly, active devices include high-frequency stirrers, pulse pressure generators, pumps, and electromagnetic generators, and passive devices are typically realized based on shape design of microchannels.

Such passive devices can move fluids using capillary flow. Capillary flow is a phenomenon where liquid flow is generated by surface tension between a liquid surface and a solid surface when liquid contacts a solid surface of a channel such as a capillary. Since capillary flow is generated by natural force due to the physical properties of liquids and solids, liquid flow can be generated without using external control. A microfluidic control device includes a microchannel, whose high surface area-to-volume ratio is a very important factor that allows the microchannel to induce liquid flow using capillary force.

### SUMMARY OF THE INVENTION

The present invention provides a device configured to dilute microfluids in a predetermined dilution ratio.

The present invention also provides a device configured to dilute microfluids in a predetermined dilution ratio with securing productivity, by simply dropping specimens and dilutions without using external control since capillary force is used as driving force.

The present invention also provides a device including dilution parts that are formed in multi stages so as to obtain a desired dilution ratio.

The present invention also provides a device that includes various chambers storing fluids, e.g., a fluid chamber and a confluence chamber that are provided with components for biological reaction and detection, so as to realize a biochip.

The present invention also provides a device functioning as various chemical reactors that require dilution ratio variation.

Embodiments of the present invention provide microfluidic dilution devices including: a cover plate; and a channel plate coupled to the cover plate and including a microfluidic dilution part. The microfluidic dilution part includes: a first fluid chamber in which a first fluid is supplied and stored; a second fluid chamber in which a second fluid is supplied and stored, the second fluid chamber having a predetermined flow resistance ratio to the first fluid chamber; a first microchannel having an end connected to a side of the first fluid chamber; a second microchannel having an end connected to another side of the first fluid chamber; a third microchannel having an end connected to a side of the second fluid chamber, and another end connected to another end of the first microchannel to provide a first confluence point; a fourth microchannel having a first end connected to another side of the second fluid chamber, and a second end connected to another end of the second microchannel to provide a second confluence point; and a micro mixer connected to the second confluence point.

In some embodiments, the second fluid chamber may be filled with the second fluid by a capillary force, and the second fluid may move to the first and second confluence points, and the first fluid may be sequentially moved from the first confluence point through the first fluid chamber to the second confluence point by the capillary force, and join the second fluid at the second confluence point in a predetermined ratio according to a flow resistance ratio of the first fluid chamber and the second fluid chamber, and be mixed with the second fluid at the micro mixer.

In other embodiments, the microfluidic dilution devices may further include: a first fluid storage connected to the first confluence point to supply the first fluid; and a second fluid storage supplying the second fluid to the first end of the fourth microchannel through a flow resistance channel.

In still other embodiments, the flow resistance channel may be greater than the first through fourth microchannels in flow resistance. The flow resistance channel may be about one-tenth or less than each of the first through fourth microchannels in width or height. The flow resistance channel may be about ten or more times greater than each of the first through fourth microchannels in length.

In even other embodiments, the microfluidic dilution devices may further include a confluence chamber connected to the micro mixer and storing the first and second fluids in a mixed state. The confluence chamber may be equal to or less than a sum of the first and second fluid chambers, in capacity. The confluence chamber may include therein at least one of antigens, antibodies, enzymes, micro/nano particles, electrodes, and sensors for biological reaction and detection of the first fluid in a diluted state.

In yet other embodiments, the first and second confluence points may be constructed by abruptly expanding the width of channel, so that the microchannels rapidly expand in the capillary flow directions at the confluence points to increase a capillary stop pressure. The first and second confluence points may include channels, surfaces of which are treated to be hydrophobic to increase a capillary stop pressure.

In further embodiments, the first fluid chamber may be extended between the first microchannel and the second microchannel, and the second fluid chamber may be extended between the third microchannel and the fourth microchannel. The first and second fluid chambers may be different in width or height.

In still further embodiments, at least one of the first and second fluid chambers may include therein at least one of antigens, antibodies, enzymes, micro/nano particles, electrodes, and sensors for biological reaction and detection.

In even further embodiments, the micro mixer may include a serpentine microchannel to mix the first and second fluids that join each other at the second confluence point, or may include a three-dimensional fluid stirrer.

In yet further embodiments, the first and third microchannels, connected to each other at the first confluence point, may be symmetrical with respect to the first confluence point, and the second and fourth microchannels, branched from the second confluence point, may be symmetrical with respect to the second confluence point.

In much further embodiments, the microfluidic dilution part may have a depth of about 100 μm or less.

In still much further embodiments, the microfluidic dilution part may be provided in plurality, and the microfluidic dilution parts may include fluid inlet holes through which different fluids are supplied, respectively.

### BRIEF DESCRIPTION OF THE FIGURES

The accompanying figures are included to provide a further understanding of the present invention, and are incorporated in and constitute a part of this specification. The drawings illustrate exemplary embodiments of the present invention and, together with the description, serve to explain principles of the present invention. In the figures:

FIG. 1 is an exploded perspective view illustrating a microfluidic dilution device according to an embodiment of the present invention;

FIG. 2 is a schematic view illustrating components of a channel plate of the microfluidic dilution device illustrated in FIG. 1;

FIG. 3 is a plan view illustrating a channel plate to which the components of FIG. 2 are applied;

FIG. 4A is an enlarged view illustrating a portion S1 of FIG. 3;

FIG. 4B is an enlarged view illustrating a portion S3 of FIG. 4A;

FIG. 5A is an enlarged view illustrating a portion S2 of FIG. 3;

FIG. 5B is an enlarged view illustrating a portion S4 of FIG. 5A;

FIGS. 6A through 6H are plan views illustrating a process of diluting a first fluid with a second fluid at the microfluidic dilution device illustrated in FIG. 1;

FIG. 7 is a schematic view illustrating a microfluidic dilution device configured to vary a dilution ratio by varying flow resistances, according to an embodiment of the present invention; and

FIG. 8 is a schematic view illustrating a microfluidic dilution device including microfluidic dilution parts for varying a dilution ratio, according to an embodiment of the present invention.

### DETAILED DESCRIPTION OF PREFERRED EMBODIMENTS

Preferred embodiments of the present invention will be described below in more detail with reference to the accompanying drawings. The present invention may, however, be embodied in different forms and should not be constructed as limited to the embodiments set forth herein. Rather, these embodiments are provided so that this disclosure will be thorough and complete, and will fully convey the scope of the present invention to those skilled in the art. Like reference numerals refer to like elements throughout.

In the present invention, both ‘dilute’ and ‘mix’ mean to combine at least two fluids.

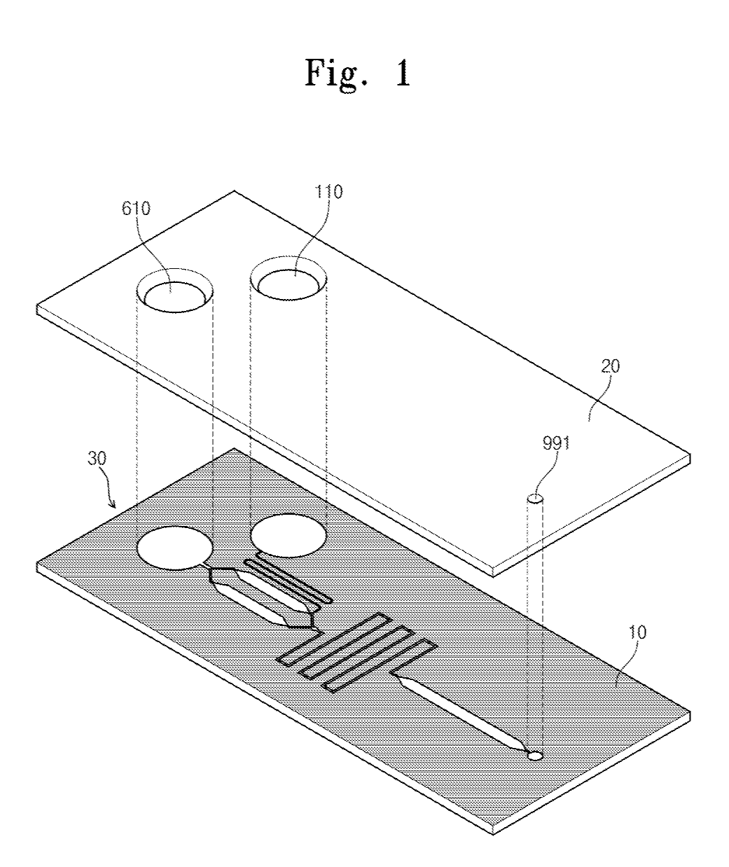

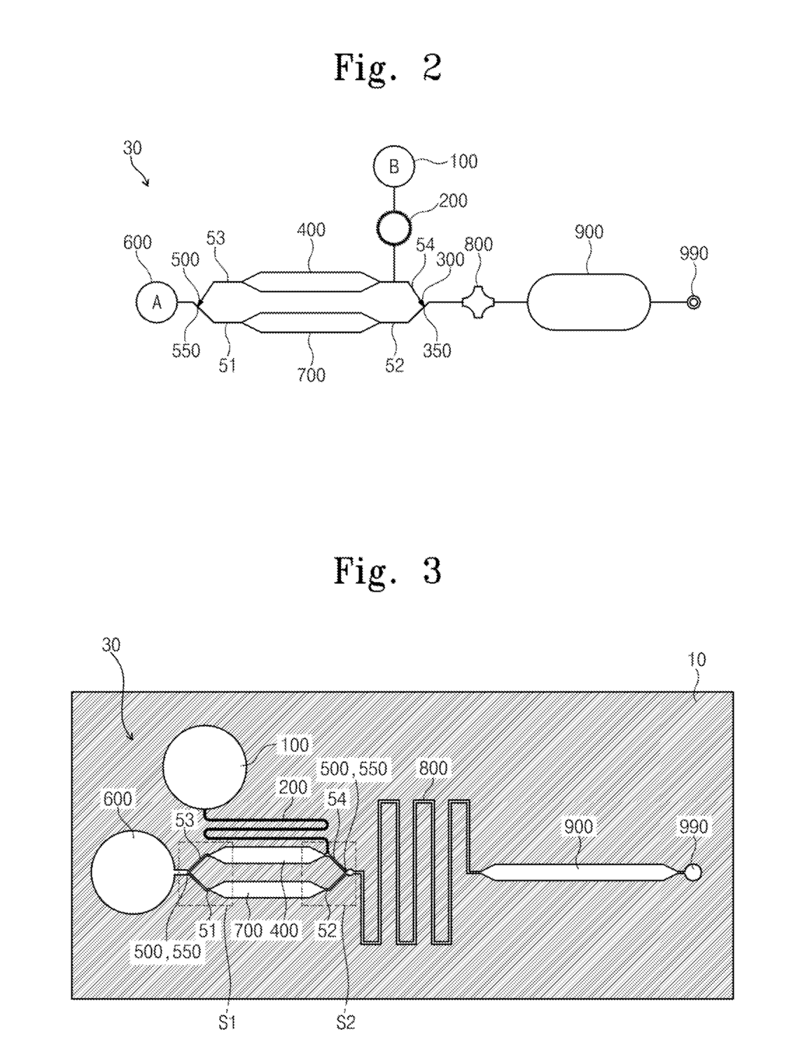

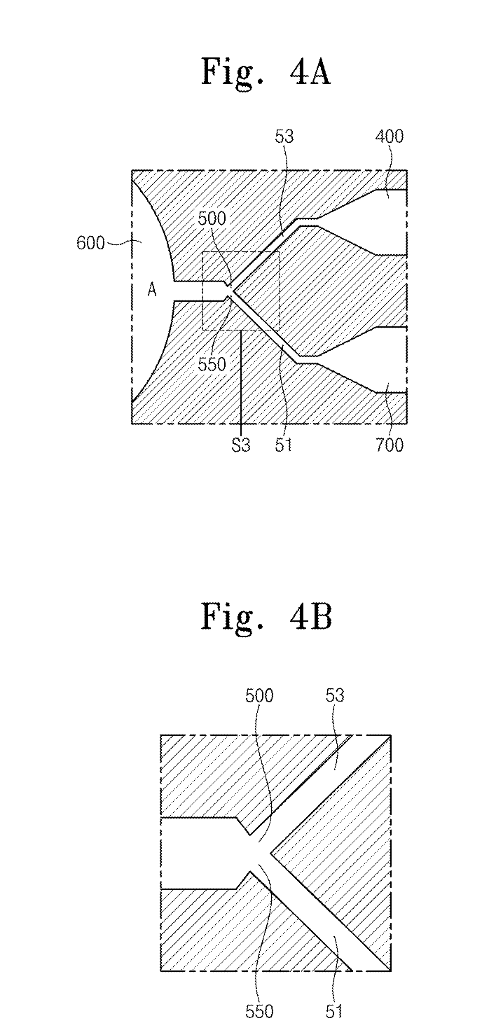

FIG. 1 is an exploded perspective view illustrating a microfluidic dilution device according to one embodiment of the present invention. FIG. 2 is a schematic view illustrating components of a channel plate 10 of the microfluidic dilution device illustrated in FIG. 1. FIG. 3 is a plan view illustrating the components of FIG. 2 applied to the channel plate 10.

The microfluidic dilution device is used to dilute a plurality of fluids, e.g. a specimen and a dilution in a predetermined ratio. Hereinafter, a plurality of fluids are referred to as a first fluid and a second fluid, but the type or the number thereof is not limited thereto.

Referring to FIGS. 1 through 3, the microfluidic dilution device includes a cover plate 20 and the channel plate 10 facing the cover plate 20 and bonded to the cover plate 20.

At least one of the channel plate 10 and the cover plate 20 may be formed of any one of polymer, silicon, glass, and a combination thereof.

The cover plate 20 is provided with a first fluid inlet hole 610 and a second fluid inlet hole 10 that provide a first fluid A and a second fluid B to be diluted. Also, an air hole 991 is provided to the cover plate 20.

The first fluid inlet hole 610 and the second fluid inlet hole 110 pass through the cover plate 20, and the first fluid A and the second fluid B are supplied to the channel plate 10 through the first fluid inlet hole 610 and the second fluid inlet hole 110.

The channel plate 10 is provided with channels through which microfluids move, and includes a microfluidic dilution part 30 for diluting a plurality of microfluids.

The microfluidic dilution part 30 may include a first fluid storage 600, a second fluid storage 100, a first fluid chamber 700, a second fluid chamber 400, a micro mixer 800, a confluence chamber 900 connected to the micro mixer 800, and an air hole part 990 connected to the confluence chamber 900.

The first fluid storage 600 and the second fluid storage 100 receive the first fluid A and the second fluid B from an outside and store them temporarily. Thereafter, the first fluid storage 600 and the second fluid storage 100 respectively supply the first fluid A and the second fluid B to other components of the microfluidic dilution part 30 to dilute the first fluid A and the second fluid B in the components of the microfluidic dilution part 30.

The first fluid storage 600 is connected to the first fluid chamber 700 through a first microchannel 51. That is, a first end of the first microchannel 51 is connected to a first end of the first fluid chamber 700, and a second end of the first microchannel 51 is connected to the first fluid storage 600. A second end of the first fluid chamber 700 is connected to a first end of a second microchannel 52.

The first fluid chamber 700 may be extended between the first microchannel 51 and the second microchannel 52. That is, the length of the first fluid chamber 700 in a capillary flow direction may be greater than the width thereof for a reliable flow via capillary force.

The second fluid chamber 400 has a predetermined flow resistance ratio to the first fluid chamber 700. The flow resistance ratio affects a flow rate. Thus, under the same driving pressure, when a flow resistance is large, the flow rate is small, and vice versa. Since the flow resistance depends on a cross-sectional area, the flow resistance ratio can be adjusted by varying the cross-sectional area of a fluid chamber (the width of a fluid chamber×the depth thereof). For example, when fluid chambers are the same in cross-sectional area and length, and thus when the flow resistances thereof are the same, their flow rates are also the same, so that a mix/dilution volume ratio is 1:1. When the fluid chambers are different in cross-sectional area and length, and thus when the flow resistance ratio is changed to a predetermined ratio (e.g. 1:2), the flow ratio corresponds to the predetermined ratio, so that the mix/dilution volume ratio is a predetermined ratio (2:1). In the present embodiment, the flow resistance of the second fluid chamber 400 is the same as that of the first fluid chamber 700.

The second fluid chamber 400 is connected to the first fluid storage 600 through a third microchannel 53. That is, a first end of the third microchannel 53 is connected to a first end of the second fluid chamber 400, and a second end of the third microchannel 53 is connected to the first fluid storage 600. A second end of the second fluid chamber 400 is connected to a first end of a fourth microchannel 54.

The second fluid chamber 400 may be extended between the third microchannel 53 and the fourth microchannel 54. That is, the length of the second fluid chamber 400 in a capillary flow direction may be greater than the width thereof for a reliable flow via capillary force.

The second end of the third microchannel 53 joins the second end of the first microchannel 51 at a first confluence point 550 to be connected to the first fluid storage 600. The first confluence point 550 is provided with a first stop valve 500 that prevents, under a predetermined condition, a fluid from flowing to the microchannel 51 or to the first fluid storage 600 from the third microchannel 53.

For the first stop valve 500 to increase a capillary stop pressure at the first confluence point 550, the second end of the third microchannel 53 may rapidly expand at the first confluence point 550. In the same manner, for a second stop valve 300 to increase a capillary stop pressure at a second confluence point 350, a second end of the fourth microchannel 54 may rapidly expand at the second confluence point 350.

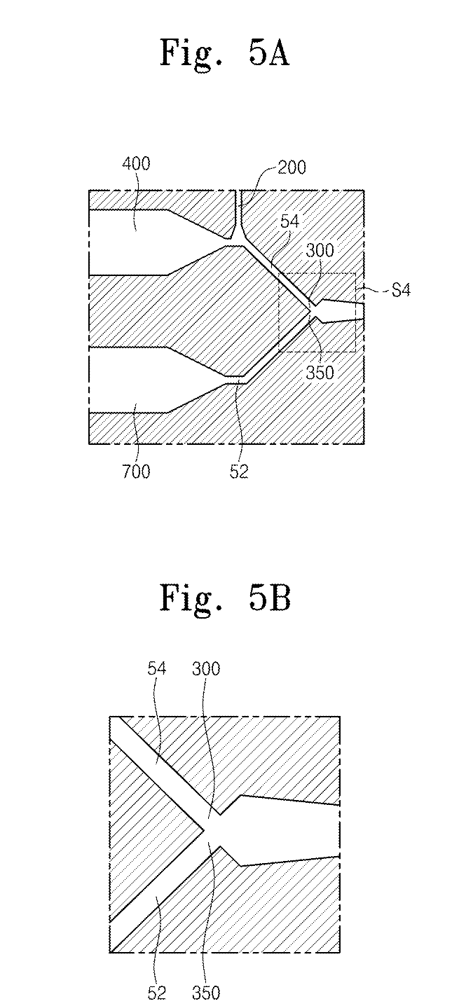

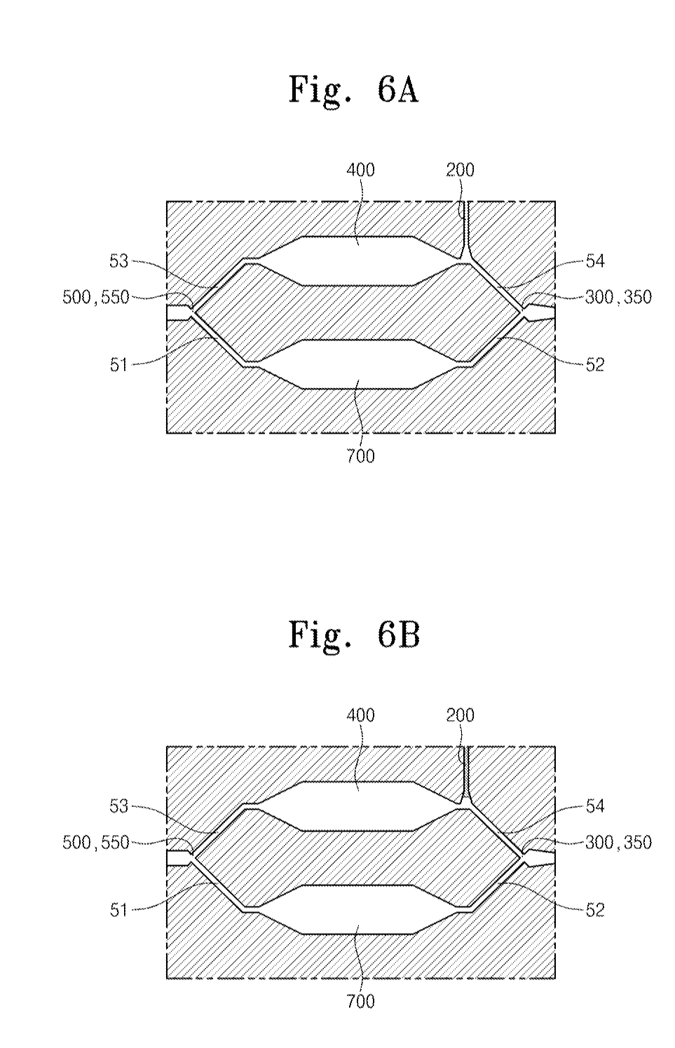

FIG. 4A is an enlarged view illustrating a portion S1 of FIG. 3, and FIG. 4B is an enlarged view illustrating a portion S3 of FIG. 4A, which illustrate the first stop valve 500.

Referring to FIGS. 4A and 4B, the first microchannel 51 joins the third microchannel 53 at the first confluence point 550 that is connected to the first fluid storage 600. The width of the second end of the third microchannel 53 rapidly expands at the first confluence point 550 such that the width of a channel in the first confluence point 550 is greater than those of the first microchannel 51 and the third microchannel 53. As such, when the width of the channel rapidly expands in the first confluence point 550, the capillary stop pressure is increased, and thus flow due to the capillary force is suppressed. As a result, a fluid flowing from the third microchannel 53 is stopped. Accordingly, the first confluence point 550 functions as the first stop valve 500. The first and third microchannels 51 and 53 branched from the first confluence point 550 are symmetrical with respect to the first confluence point 550, so that the flow resistances thereof are the same.

To realize the first stop valve 500, while the exemplified channel in the first confluence point 550 expands rapidly, the surface of the first confluence point 550 may be treated to be hydrophobic in other embodiments. When the surface is hydrophobic, and when a contact angle between a fluid and the surface is about 90 degrees or more, the surface tends to push out the fluid, which realizes the first stop valve 500.

The second end of the fourth microchannel 54 joins a second end of the second microchannel 52 at the second confluence point 350 to be connected to the micro mixer 800. The second confluence point 350 is provided with the second stop valve 300 that prevents, under a predetermined condition, a fluid from flowing to the second microchannel 52 or to the micro mixer 800 from the fourth microchannel 54.

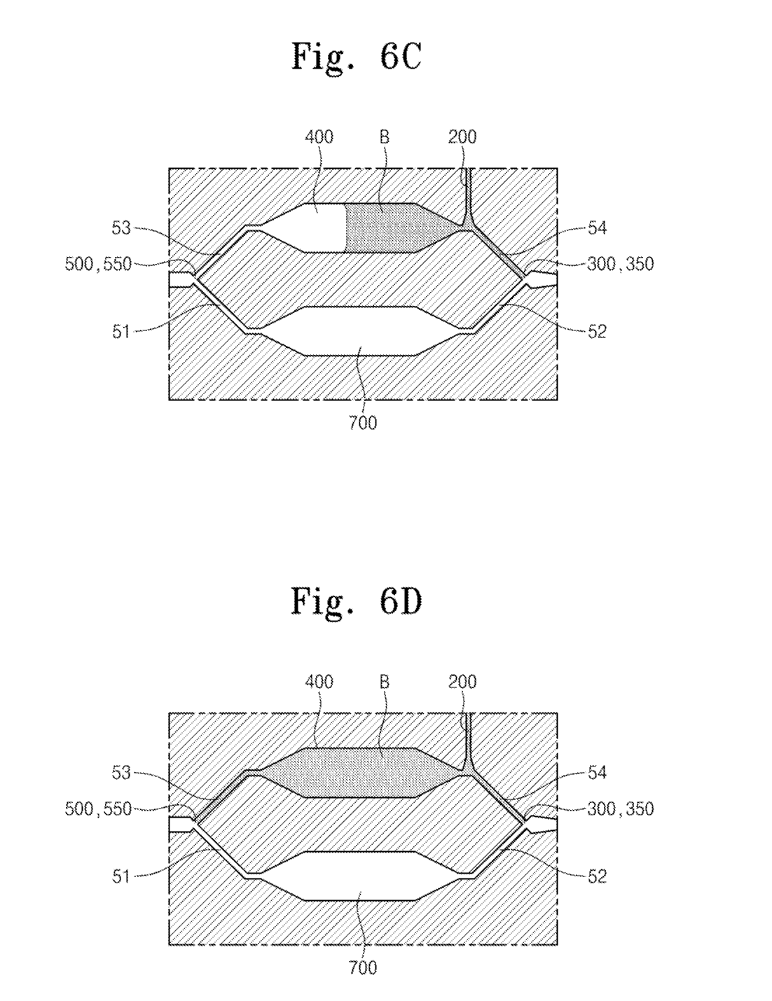

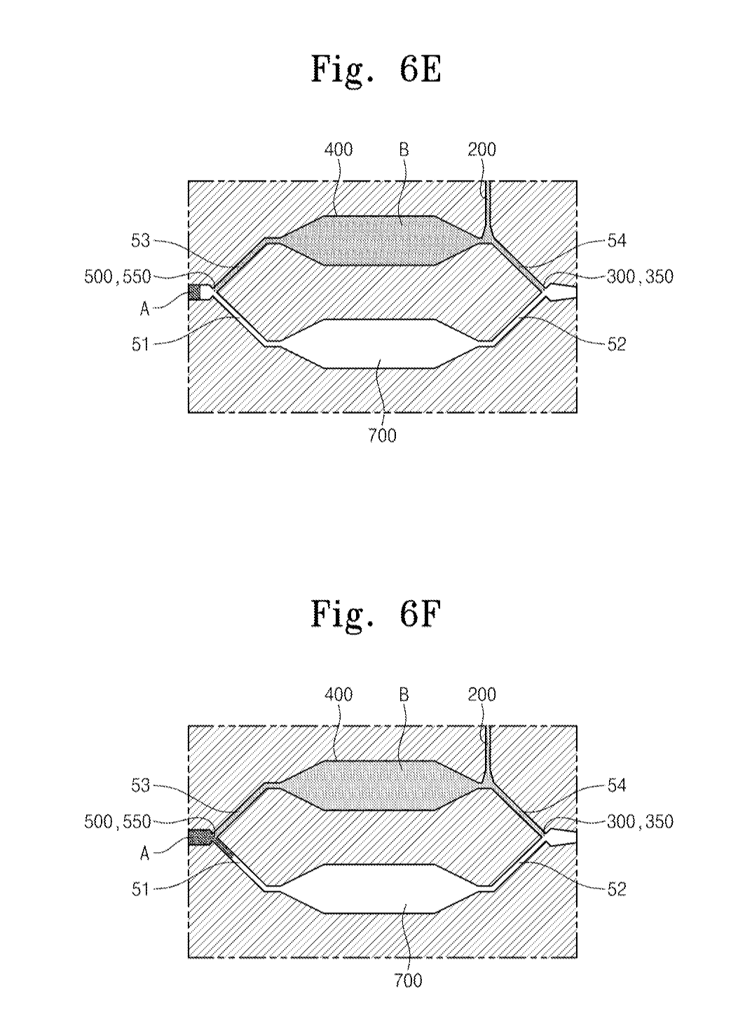

FIG. 5A is an enlarged view illustrating a portion S2 of FIG. 3, and FIG. 5B is an enlarged view illustrating a portion S4 of FIG. 5A, which illustrate the second stop valve 300.

Referring to FIGS. 5A and 5B, the second microchannel 52 joins the fourth microchannel 54 at the second confluence point 350 that is connected to the micro mixer 800. The width of the second end of the fourth microchannel 54 rapidly expands at the second confluence point 350 such that the width of a channel in the second confluence point 350 is greater than those of the second microchannel 52 and the fourth microchannel 54. As such, in the same manner as the first stop valve 500, the second confluence point 350 prevents a fluid from flowing from the fourth microchannel 54, and thus functions as the second stop valve 300. The second and fourth microchannels 52 and 54 branched from the second confluence point 350 are symmetrical with respect to the second confluence point 350, so that the flow resistances thereof are the same so as to obtain an accurate dilution ratio.

It will be appreciated that the surface of the second stop valve 300 may be also treated to be hydrophobic in other embodiments in order to realize the second stop valve 300.

Referring to FIGS. 1 through 3, the first and third microchannels 51 and 53 connected at the first confluence point 550 are symmetrical to each other with respect to the first confluence point 550. The second and fourth microchannels 52 and 54 connected at the second confluence point 350 are symmetrical to each other with respect to the second confluence point 350. Accordingly, when the microfluidic dilution device is driven, a dilution ratio of the first fluid A and the second fluid B joining the second confluence point 350 is 1:1. As such, fluids passing respectively through symmetrical channels have the same flow rate, and thus the same amounts of the fluids are mixed at a confluence point.

The first end of the fourth microchannel 54 is connected to the second fluid storage 100 through a flow resistance channel 200.

The flow resistance channel 200 is provided with a microchannel such that the capillary force moves the second fluid B. The flow resistance of the flow resistance channel 200 is much greater than those of the first through fourth microchannels 51, 52, 53, and 54.

This is for preventing, under a predetermined condition, the second fluid B from flowing through the flow resistance channel 200. While filling the second fluid chamber 400 with the second fluid B, the second fluid B flows to the second fluid chamber 400 and the fourth microchannel 54 from the second fluid storage 100 through the flow resistance channel 200. However, when the first and second fluids A and B joined at the second confluence point 350 flow to the confluence chamber 900 through the micro mixer 800, the flow resistance of the flow resistance channel 200 is much greater than that of the second fluid chamber 400 and that of the third microchannel 53, and thus, the second fluid B flows rather from the second fluid chamber 400 through the fourth microchannel 54 to the micro mixer 800 than from the flow resistance channel 200 through the fourth microchannel 54 to the micro mixer 800.

According to one embodiment of the present invention, the width or height of the flow resistance channel 200 may be one-tenth or less of the respective widths or heights of the first through fourth microchannels 51, 52, 53, and 54 to increase the flow resistance of the flow resistance channel 200. Alternatively, according to another embodiment, the length of the flow resistance channel 200 may be ten or more times greater than the respective lengths of the first through fourth microchannels 51, 52, 53, and 54.

A first end of the micro mixer 800 is connected to the second confluence point 350. The micro mixer 800, for mixing the first fluid A and the second fluid B joining the second confluence point 350, includes a bent microchannel to mix the first fluid A and the second fluid B. In another embodiment of the present invention, a three-dimensional fluid stirrer may be used as the micro mixer 800.

A second end of the micro mixer 800 is connected to the confluence chamber 900 storing the mixed first and second fluids A and B.

To store the mixed first and second fluids A and B, the capacity of the confluence chamber 900 is equal to the sum of the capacities of the first fluid chamber 700 and the second fluid chamber 400, or less than the sum.

The confluence chamber 900 is provided with the air hole part 990 that is configured to discharge air from microchannels. The cover plate 20 is provided with the air hole 991 that communicates with the air hole part 990 to discharge air from the confluence chamber 900.

The components of the microfluidic dilution part 30 including the first through fourth microchannels 51, 52, 53, and 54 have depths of about 100 μm or less. Thus, the capillary force controls microfluids.

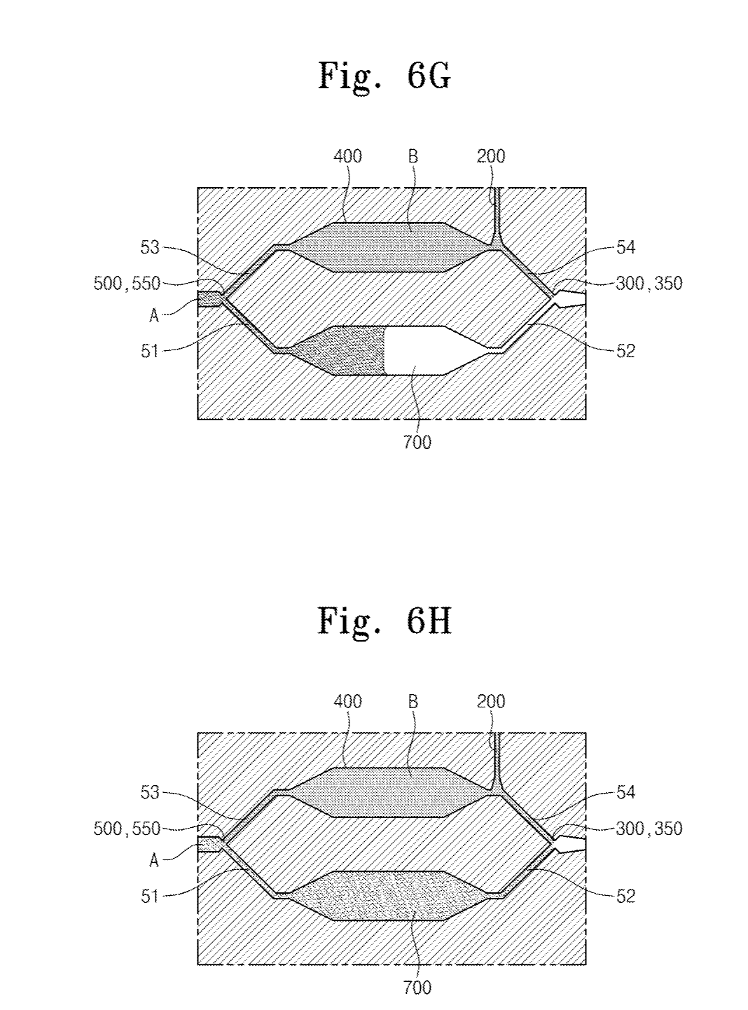

FIGS. 6A through 6H are plan views illustrating a process of driving the microfluidic dilution device according to the embodiment of FIG. 1, that is, a process of diluting the first fluid A with the second fluid B, in which the microfluidic dilution part 30 is partially shown.

Referring to FIGS. 1 through 3, and FIGS. 6A through 6H, the diluting process according to the embodiment of FIG. 1 will now be described.

The second fluid B is supplied to the second fluid inlet hole 110. The capillary force fills the flow resistance channel 200 with the supplied second fluid B (refer to FIG. 6B). Continuously, the capillary force fills the second fluid chamber 400 and the fourth microchannel 54 with the second fluid B (refer to FIG. 6C). Then, the second stop valve 300 stops the second fluid B at the second confluence point 350. The capillary force continuously moves the second fluid B to fill up the second fluid chamber 400 and then fill the third microchannel 53. Then, the first stop valve 500 stops the second fluid B at the first confluence point 550 (refer to FIG. 6D).

The first fluid A is supplied to the first fluid inlet hole 610 (refer to FIG. 6E) and moved to the first confluence point 550 (refer to FIG. 6F). The capillary force fills the first fluid chamber 700 with the supplied first fluid A passing through the first microchannel 51 (refer to FIG. 6G). The first fluid A continuously moves to the second microchannel 52. When the first fluid A arrives at the first confluence point 550, the first fluid A joins the stopped second fluid B (refer to FIG. 6H). That is, the first fluid A joins the second fluid B at the first confluence point 550 through interfacial mixing after the surface of the stopped second fluid B contacts the surface of the first fluid A.

Since the first fluid A joins the second fluid B at the second confluence point 350, the second confluence point 350 does not function as the second stop valve 300 any more. Thus, from the first fluid storage 600, the first fluid A flows sequentially to the first confluence point 550, the first microchannel 51, the first fluid chamber 700, the second microchannel 52, and the second confluence point 350. At the same time, from the first fluid storage 600, a flow is successively generated to the first confluence point 550, the third microchannel 53, the second fluid chamber 400, the fourth microchannel 54, and the second confluence point 350. At this point, the high flow resistance of the flow resistance channel 200 prevents the second fluid B from flowing out from the second fluid storage 100. That is, when the capillary force moves the joined first and second fluids A and B to the micro mixer 800 and the confluence chamber 900, the flow resistance channel 200 has the greater flow resistance than the second fluid chamber 400 so as to prevent the second fluid B stored in the second fluid storage 100 from flowing into the confluence chamber 900.

Thus, the second fluid B of the second fluid chamber 400 and the first fluid A of the first fluid chamber 700 are mixed in the same volume (refer to FIG. 6I).

After that, the joined first and second fluids A and B flow through the micro mixer 800 to the confluence chamber 900 by the capillary force. Through this process, the supplied first and second fluids A and B are mixed in the predetermined dilution ratio by the capillary force and stored in the confluence chamber 900. The air hole part 990 and the air hole 991 exhaust air such that the capillary force efficiently makes the flow.

Through the driving of the microfluidic dilution device as described above, the first fluid A and the second fluid B are mixed in a ratio of one-to-one that corresponds to the flow resistance ratio of the first fluid chamber 700 and the second fluid chamber 400. The microfluidic dilution device can be used for biological reaction and detection. For example, the inner space of at least one of the first fluid chamber 700, the second fluid chamber 400, and the confluence chamber 900 may be provided with at least one of antigens, antibodies, enzymes, micro/nano particles, electrodes, and sensors for the biological reaction and detection of the first fluid A, the second fluid B, and the mixture thereof.

As described above, the microfluidic dilution device is used to mix first and second fluids in a ratio of one-to-one. According to another embodiment of the present invention, provided is a microfluidic dilution device for mixing first and second fluids in a ratio that is different from the ratio of one-to-one.

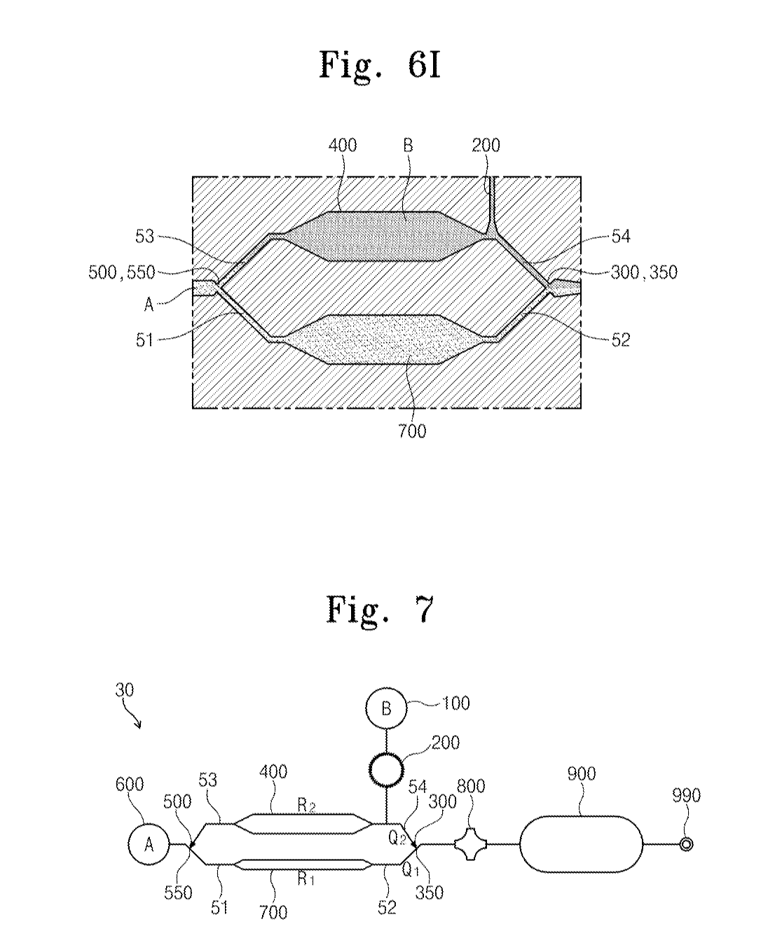

FIG. 7 is a schematic view illustrating the microfluidic dilution device according to the present embodiment, which is configured to vary a dilution ratio by varying flow resistances.

Referring to FIG. 7, the sizes of the first fluid chamber 700 and the second fluid chamber 400 are different in order to make flow resistances R₁ and R₂ that are different from each other while the first fluid A and the second fluid B flow to the micro mixer 800 and the confluence chamber 900. Since a flow rate is in inverse proportional to a flow resistance under the same flow pressure, a flow rate ratio is expressed as Equation (1).

Q₁/Q₂=R₂/R₁ (1)
where Q₁ denots the flow rate of the first fluid A, and Q₂ denots the flow rate of the second fluid B.

For example, when the flow resistance R₁ of the first fluid chamber 700 is the same as the flow resistance R₂ of the second fluid chamber 400, the flow rate ratio of the first and second fluids A and B flowing to the confluence chamber 900 is 1:1, and when the flow resistance R₁ of the first fluid chamber 700 is 2 times greater than the flow resistance R₂ of the second fluid chamber 400, the flow rate ratio of the first and second fluids A and B flowing to the confluence chamber 900 is 1:2.

The first fluid chamber 700 and the second fluid chamber 400 may be different in width and height.

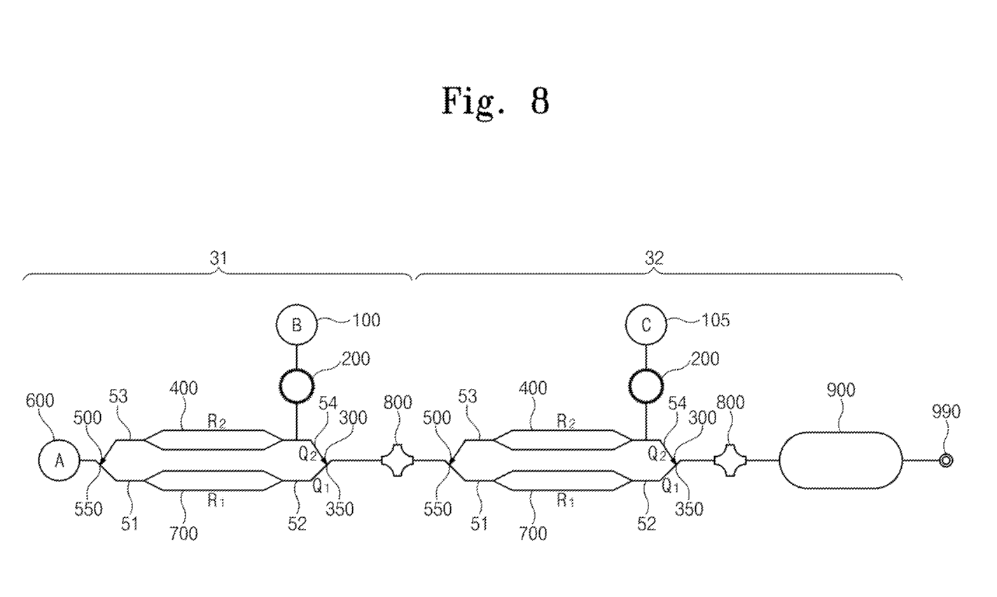

FIG. 8 is a schematic view illustrating a microfluidic dilution device including first and second microfluidic dilution parts 31 and 32 for varying a dilution ratio, according to another embodiment of the present invention. According to the present embodiment, three or more fluids can be mixed or diluted in a predetermined ratio.

Referring to FIG. 8, the micro mixer 800 of the first microfluidic dilution part 31 is connected to the first confluence point 550 of the second microfluidic dilution part 32.

The first and second fluids A and B are supplied to the first microfluidic dilution part 31, and a third fluid C to be mixed is supplied to the second microfluidic dilution part 32 through a third fluid storage 105. Thus, the first fluid A and the second fluid B are mixed in a predetermined ratio through the first microfluidic dilution part 31, and the mixture thereof is mixed with the third fluid C through the second microfluidic dilution part 32.

When the first and second fluid chambers 700 and 400 of the first microfluidic dilution part 31 are equal in flow resistance, and when the first and second fluid chambers 700 and 400 of the second microfluidic dilution part 32 are equal in flow resistance, the first fluid A and the second fluid B are mixed in a ratio of 1:1 at the first microfluidic dilution part 31, and then the mixture of the first fluid A and the second fluid B is mixed with the third fluid C in a ratio of 1:1 at the second microfluidic dilution part 32. Thus, the first fluid A, the second fluid B and the third fluid C in the final mixture are mixed in a ratio of 1:1:2. When the second fluid B are the same as the third fluid C, the first fluid A and the second fluid B in the final mixture are mixed in a ratio of 1:3.

In the same manner, when a microfluidic dilution device is provided with n stages in which the first fluid chamber 700 and the second fluid chamber 400 are equal in flow resistance, and when all fluids except for the first fluid A are the second fluid B, the volume ratio of the first fluid A and the second fluid B stored in the final confluence chamber 900 is expressed as Equation (2).

V_{B}/V_{A}=2ⁿ−1 (2)
where V_{A} denots the volume of the first fluid A, and V_{B} denots the volume of the second fluid B.

As described above, the microfluidic dilution device according to the present invention dilutes microfluids in a predetermined ratio, and external control is not required since the capillary force is used as a driving force.

In addition, microfluids can be mixed in a predetermined ratio with securing productivity, by simply dropping specimens and dilutions, and a desired dilution ratio can be obtained by arranging a plurality of microfluidic dilution parts.

In addition, according to the present invention, a specimen fluid chamber, a dilution fluid chamber, and a confluence chamber are provided with components for biological reaction and detection to realize a biochip and various chemical reactors that require dilution ratio variation.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A microfluidic dilution device comprising:
a cover plate; and
a channel plate bonded to the cover plate and including a microfluidic dilution part,
wherein the microfluidic dilution part includes:
a first fluid chamber in which a first fluid is supplied and stored;
a second fluid chamber in which a second fluid is supplied and stored, the second fluid chamber having a predetermined flow resistance ratio to the first fluid chamber;
a first microchannel having an end connected to a side of the first fluid chamber;
a second microchannel having an end connected to another side of the first fluid chamber;
a third microchannel having an end connected to a side of the second fluid chamber, and another end connected to another end of the first microchannel to provide a first confluence point;
a fourth microchannel having a first end connected to another side of the second fluid chamber, and a second end connected to another end of the second microchannel to provide a second confluence point; and
a micro mixer connected to the second confluence point.

2. The microfluidic dilution device of claim 1, wherein the second fluid chamber is filled with the second fluid by a capillary force, and the second fluid moves to the first and second confluence points, and
the first fluid is sequentially moved from the first confluence point through the first fluid chamber to the second confluence point by the capillary force, and joins the second fluid at the second confluence point in a predetermined ratio according to a flow resistance ratio of the first fluid chamber and the second fluid chamber, and is mixed with the second fluid at the micro mixer.

3. The microfluidic dilution device of claim 1, further comprising:
a first fluid storage connected to the first confluence point to supply the first fluid; and
a second fluid storage supplying the second fluid to the first end of the fourth microchannel through a flow resistance channel.

4. The microfluidic dilution device of claim 3, wherein the flow resistance channel is greater than the first through fourth microchannels in flow resistance.

5. The microfluidic dilution device of claim 4, wherein the flow resistance channel is one-tenth or less than each of the first through fourth microchannels in width or height.

6. The microfluidic dilution device of claim 4, wherein the flow resistance channel is ten or more times greater than each of the first through fourth microchannels in length.

7. The microfluidic dilution device of claim 1, further comprising a confluence chamber connected to the micro mixer and storing the first and second fluids in a mixed state.

8. The microfluidic dilution device of claim 7, wherein the confluence chamber is equal to or less than a sum of the first and second fluid chambers, in capacity.

9. The microfluidic dilution device of claim 7, wherein the confluence chamber comprises therein at least one of antigens, antibodies, enzymes, micro/nano particles, electrodes, and sensors for biological reaction and detection of the first fluid in a diluted state.

10. The microfluidic dilution device of claim 1, wherein the first and second confluence points is constructed by abruptly expanding the width of channel, so that the microchannels rapidly expand in the capillary flow directions at the first and second confluence points to increase a capillary stop pressure.

11. The microfluidic dilution device of claim 1, wherein the first and second confluence points comprise channels, surfaces of which are treated to be hydrophobic to increase a capillary stop pressure.

12. The microfluidic dilution device of claim 1, wherein the first fluid chamber is extended between the first microchannel and the second microchannel, and the second fluid chamber is extended between the third microchannel and the fourth microchannel.

13. The microfluidic dilution device of claim 12, wherein the first and second fluid chambers are different in width or height.

14. The microfluidic dilution device of claim 1, wherein at least one of the first and second fluid chambers comprise therein at least one of antigens, antibodies, enzymes, micro/nano particles, electrodes, and sensors for biological reaction and detection.

15. The microfluidic dilution device of claim 1, wherein the micro mixer comprises a serpentine microchannel to mix the first and second fluids that join each other at the second confluence point.

16. The microfluidic dilution device of claim 1, wherein the micro mixer comprises a three-dimensional fluid stirrer configured to mix the first and second fluids that join each other at the second confluence point.

17. The microfluidic dilution device of claim 1, wherein the first and third microchannels, connected to each other at the first confluence point, are symmetrical with respect to the first confluence point, and
the second and fourth microchannels, branched from the second confluence point, are symmetrical with respect to the second confluence point.

18. The microfluidic dilution device of claim 1, wherein the microfluidic dilution part has a depth of 100 μm or less.

19. The microfluidic dilution device of claim 1, wherein the microfluidic dilution part is provided in plurality.

20. The microfluidic dilution device of claim 19, wherein the microfluidic dilution parts comprise fluid inlet holes through which different fluids are supplied, respectively.
